(19) United States
(12) Patent Application Publication
(10) Pub. No.: US 2016/0296743 A1
(43) Pub. Date: Oct. 13, 2016

(54) ELECTRICAL TREATMENT PROBE FOR CAVAL SPHINCTER

(71) Applicant: CHI-CHENG CHANG, TAICHUNG CITY (TW)

(72) Inventor: CHI-CHENG CHANG, TAICHUNG CITY (TW)

(21) Appl. No.: 14/867,571

(22) Filed: Sep. 28, 2015

(30) Foreign Application Priority Data
Apr. 9, 2015 (TW) 104205280

(51) Int. Cl. A61N 1/05

(52) U.S. Cl. CPC A61N 1/05

(57) ABSTRACT

An electrical treatment probe for caval sphincter includes: a head portion with a probe-head internal connecting tube connected to the rear of the head portion; a middle portion including a hollow middle-section tube with a first internal connecting tube and a second internal connecting tube disposed at the front and rear of the middle-section tube respectively; a tail portion including a hollow probe-tail tube with a probe-tail internal connecting tube disposed at the front of the probe-tail tube; two conductive rings sheathed on the probe-head internal connecting tube and the first internal connecting tube, and the second internal connecting tube and the probe-tail internal connecting tube respectively; a conductive wire module including first and second conductive wires which are pressed by the conductive rings to produce a contacting and positioning effect. Thus, an electrical treatment probe casing is formed by the aforementioned elements and filled with a filler.

### TECHNICAL FIELD

The technical field relates to a probe structure, and more particularly to an electrical treatment probe that performs electrical treatments through biofeedback, and the probe has the features of easy manufacture and good texture.

### BACKGROUND

At present, electrical stimulations for medical treatments are mainly used for the purposes of reducing pain, preventing and delaying muscular atrophy, relieving muscle cramp, and promoting blood circulation, and urinary incontinence generally happens to middle-aged and elderly women. For clinical treatments, electric simulation is usually applied to genital muscles to contract the urethral sphincter and train the sphincter to reduce the symptoms of urinary incontinence. For example, the causes of stress incontinence include relaxation and atrophy of pelvic muscles and abnormal atrophy of mucosa of urethral muscle, etc, and women's pelvic muscular mucosa may become loose and weak after giving birth to babies, so that the pressure at kidney is greater than the pressure at urethra, which results in urinary incontinence. The atrophy of muscles and muscular mucosa of urinary incontinence patients may be treated and improved by biofeedback electrical treatments.

With reference to FIGS. 1 and 2 for a conventional electrical treatment probe that applies electrical stimulations to caval sphincter, the conventional electrical treatment probe 90 generally comprises a rod 91 and a handle 92, wherein two conductive rings 93, 94 are sheathed on the rod 91 and respectively and electrically coupled to two conductive wires 95, 96, and the conductive wires 95, 96 are installed in the rod 91. When the electrical treatment probe 90 is manufactured, the conductive rings 93, 94 are placed in a mold (not shown in the figure) in advance, and then the conductive wires 95, 96 are welded to the conductive rings 93, 94 respectively as shown in the figures, and each of the conductive rings 93, 94 has a welding point 931, 941 formed at a welding position of the conductive wire 95, 96; and then the rod 91 and the handle 92 in the mold are formed by injection molding, so that the conductive rings 93, 94 are combined and positioned at the external peripheral surface of the rod 91, and the conductive wires 95, 96 are embedded and positioned in the rod 91.

Although the aforementioned conventional electrical treatment probe structure can achieve the effect of assembling the components, it still has the following drawbacks. After the conductive wires 95, 96 are welded with the conductive rings 93, 94 respectively, the injection molding process is carried out in the mold, and thus one or more mold ridges 911 may be formed on surfaces on both sides of the rod 91 separately. Since the electrical treatment probe 90 is provided for inserting into a human vagina (or anus), therefore the rod 91 so formed requires a refined manufacture to eliminate the mold ridges 911 and provide smooth surfaces of the rod 91. Obviously, the manufacture of the conventional electrical treatment probe 90 is too complicated and time-consuming and is incompliant with the requirement of cost-effectiveness. In addition, the electrical treatment probe 90 is an appliance to be inserted into a cava, so that the rod 91 (or the material used for injection molding) comes with a white color or a light color to make the electrical treatment probe 90 more acceptable to users. However, the temperature of the injection molding process is very high, and the pigment of the material at the positions of welding points 931, 941 may permeate to the surface of the molded rod 91 to form one or more dirty spots 912 and cause a serious flaw to the appearance of the electrical treatment probe 90. Obviously, the conventional electrical treatment probe requires improvements. Therefore, it is a main subject for related manufactures to overcome the drawbacks and problems of the conventional electrical treatment probe structure.

In view of the aforementioned drawbacks and problems of the conventional electrical treatment probe structure, the discloser of this disclosure conducted extensive researches and experiments, and finally developed an electrical treatment probe for caval sphincter with the features of easy manufacture, high effectiveness, and good quality to overcome the drawbacks and problems of the prior art.

### SUMMARY

Therefore, it is a primary objective of this disclosure to provide an electrical treatment probe for caval sphincter, wherein the electrical treatment probe comes with a good design of the manufacturing components and assemblies to improve the convenience and cost-effectiveness of the manufacture of the electrical treatment probe and also overcomes the issue of permeating pigments of the welding material to the surface electrical treatment probe of the during the injection molding process to enhance the quality of the electrical treatment probe so formed.

To achieve the aforementioned objectives, this disclosure provides an electrical treatment probe for caval sphincter, and the electrical treatment probe comprises: a head portion, including a head covering member formed by an internal hollow tube, an arc end portion disposed at a front of the head covering member, and a probe-head internal connecting tube with a smaller diameter and coupled to a rear of the head covering member; a middle portion, including a hollow middle-section tube, and a first internal connecting tube and a second internal connecting tube with a smaller diameter and coupled to the front and rears of the middle-section respectively; a tail portion, including a hollow probe-tail tube, and a probe-tail internal connecting tube with a smaller diameter and coupled to the front of the probe-tail tube; two conductive rings, one of the conductive rings being sheathed on the probe-head internal connecting tube and the first internal connecting tube of the head covering member, and the other conductive ring being sheathed on of the second internal connecting tube and the probe-tail internal connecting tube of the tail portion; a conductive wire module, including a first conductive wire and second conductive wire, and the first conductive wire being pressed by one of the conductive rings to produce a contacting and positioning effect, and the second conductive wire being pressed by the other conductive ring to produce a contacting and positioning effect; such that an electrical treatment probe casing is formed by the aforementioned elements, and the electrical treatment probe casing includes an injection molding installed therein and filled with a filler, and after the conductive ring is installed, the electric treatment probe casing, the head covering member, the middle-section tube and the probe-tail tube constitute a smooth surface of the electrical treatment probe.

In the aforementioned assembly, the probe-head internal connecting tube of the head portion and the probe-tail internal connecting tube of the tail portion have a notch separately.

In the aforementioned assembly, the first internal connecting tube and the second internal connecting tube have a notch separately.

In the aforementioned assembly, the first conductive wire is passed into the notch of the probe-head internal connecting tube or the notch of the first internal connecting tube.

In the aforementioned assembly, the second conductive wire is passed into the notch of the second internal connecting tube or the notch of the probe-tail tube.

In the aforementioned assembly, the first conductive wire has an electrically conductive portion embedded into the probe-head internal connecting tube of the head portion or the first internal connecting tube of the middle-section tube.

In the aforementioned assembly, the second conductive wire has an electrically conductive portion embedded into the second internal connecting tube of the middle-section tube or the probe-tail internal connecting tube of the probe-tail tube.

In the aforementioned assembly, the electrical treatment probe casing includes a reinforcing member installed therein.

In the aforementioned assembly, the reinforcing member has an opening communicated with a cavity in the reinforcing member, and the cavity is filled with the filler.

This disclosure will become clearer in light of the following detailed description of an illustrative embodiment of this invention described in connection with the drawings.

### BRIEF DESCRIPTION OF THE DRAWINGS

FIG. 1 is a cross-sectional view of a conventional electrical treatment probe structure;

FIG. 2 is a perspective view of a conventional electrical treatment probe structure;

FIG. 3 is a perspective view of a first preferred embodiment of this disclosure;

FIG. 4 is an exploded view of the first preferred embodiment of this disclosure;

FIG. 5 is a sectional view of the first preferred embodiment of this disclosure;

FIG. 5a is a blowup view of a portion of the first preferred embodiment of this disclosure; and.

FIG. 6 is an exploded view of a second preferred embodiment of this disclosure.

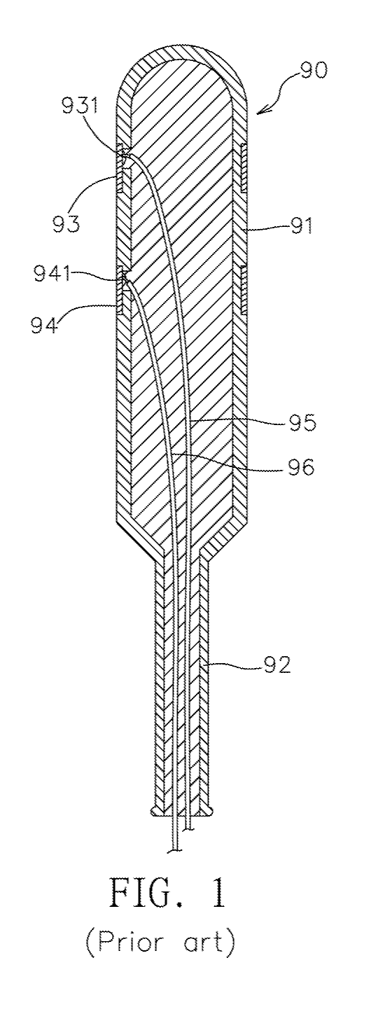

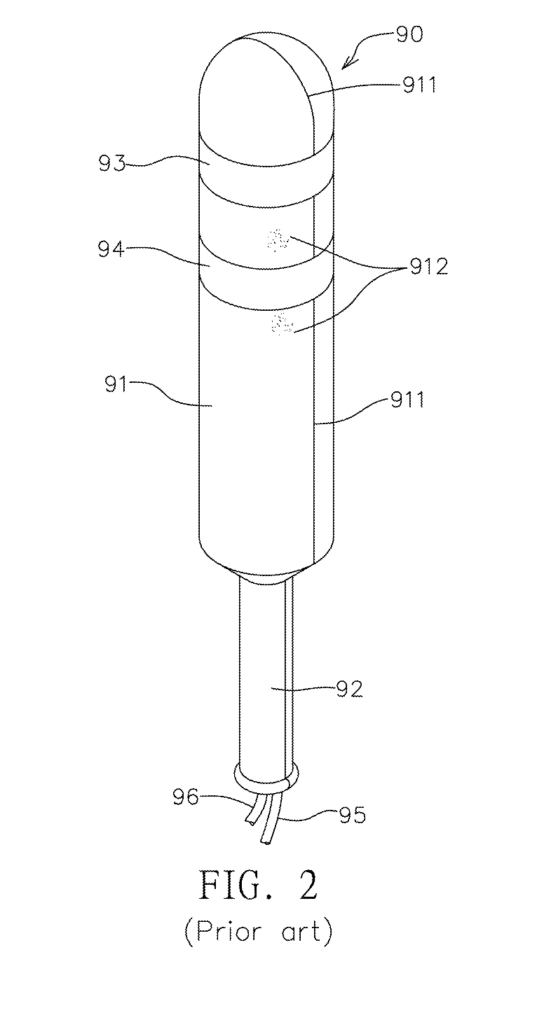

### DESCRIPTION OF THE PREFERRED EMBODIMENTS

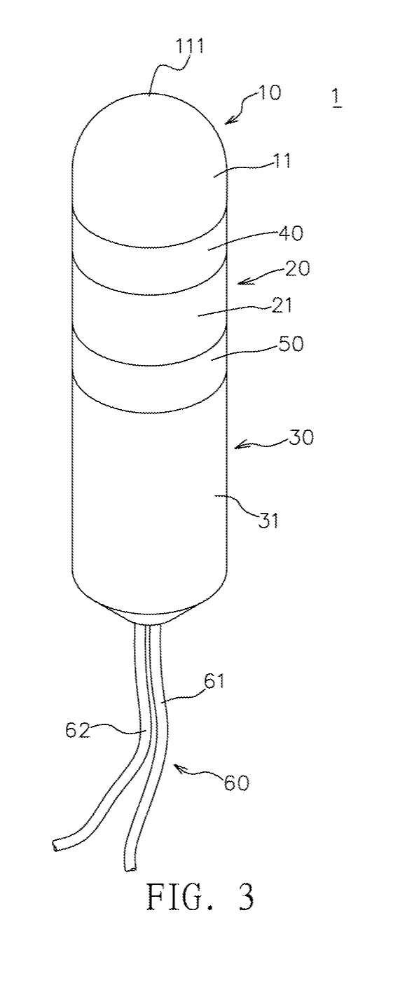

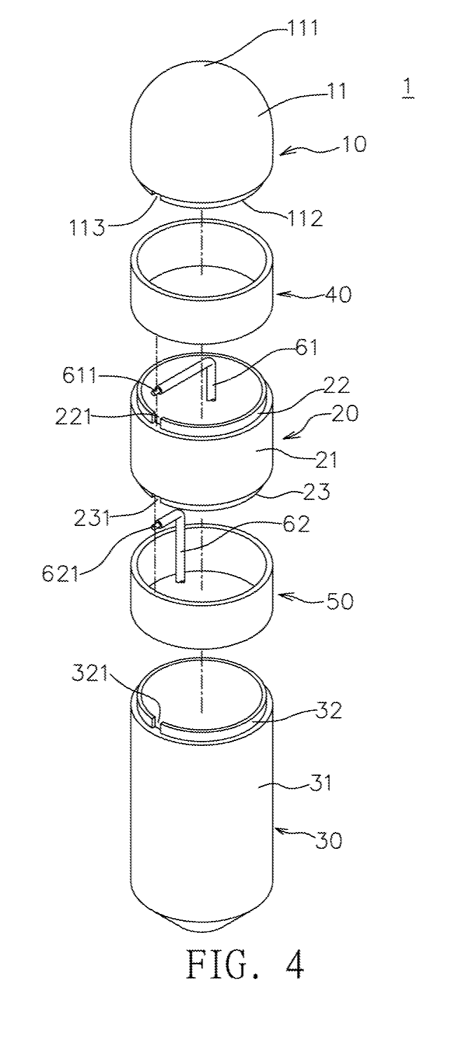

With reference to FIGS. 3, 4 and 5 for an electrical treatment probe for caval sphincter in accordance with the first preferred embodiment of this disclosure, the electrical treatment probe 1 comprises a head portion 10, a middle portion 20 and a tail portion 30. The head portion 10 includes a head covering member 11 formed by an internal hollow tube, an arc end portion 111 formed at a front of the head covering member 11 (in the direction as shown in the figure), a probe-head internal connecting tube 112 with a smaller diameter and formed at the rear of the head covering member 11 and having a notch 113. The middle portion 20 includes a middle-section tube 21 which is a hollow tube, and a first internal connecting tube 22 and a second internal connecting tube 23 with a smaller diameter and formed at the front and rear of the middle-section tube 21 respectively, wherein both of the first internal connecting tube 22 and the second internal connecting tube 23 have a notch 221, 231. The tail portion 30 is hollow inside, and includes a hollow probe-tail tube 31, and probe-tail internal connecting tube 32 with a smaller diameter and disposed at the front of the probe-tail tube 31, and the probe-tail internal connecting tube 32 has a notch 321. In this preferred embodiment, the head covering member 11, the middle-section tube 21 and the probe-tail tube 31 have substantially the same external diameter, and the probe-head internal connecting tube 112, the first internal connecting tube 22, the second internal connecting tube 23 and the probe-tail internal connecting tube 32 also have substantially the same external diameter to facilitate the manufacturing and assembling processes.

The electrical treatment probe 1 further comprises two conductive rings 40, a conductive ring 50 and a conductive wire module 60, wherein the conductive ring 40 and the conductive ring 50 are hollow metal rings, and the conductive ring 40, the conductive ring 50 and the head covering member 11 (or the middle-section tube 21 and the probe-tail tube 31) have an equal external diameter, and the conductive ring 40 and the conductive ring 50 have an internal diameter equivalent to that of the probe-head internal connecting tube 112 (or the first internal connecting tube 22, the second internal connecting tube 23 and the probe-tail internal connecting tube 32), so that after the conductive rings 40, 50 are assembled, the head covering member 11, the middle-section tube 21 and the probe-tail tube 31 give a smooth surface of the electrical treatment probe 1. The conductive ring 40 is sheathed on the probe-head internal connecting tube 112 and the first internal connecting tube 22 to position and combine the conductive ring 40 between the head covering member 11 and the middle-section tube 21, and the conductive ring 50 is sheathed on the second internal connecting tube 23 and the probe-tail internal connecting tube 32 to position and combine the conductive ring 50 between the middle-section tube 21 and the probe-tail tube 31.

The conductive wire module 60 is entered from the probe-tail tube 31 into the electrical treatment probe 1, and the conductive wire module 60 includes a first conductive wire 61 and a second conductive wire 62, and the first conductive wire 61 is passed and installed into the notch 113 (or the notch 221), so that an electrically conductive portion 611 (such as a metal conductive wire) is pressed by the conductive ring 40 to produce a contacting and positioning effect. The second conductive wire 62 is passed and installed into the notch 231 (or the notch 321), so that an electrically conductive portion 621 (such as a metal conductive wire) is pressed by the conductive ring 50 to produce a contacting and positioning effect.

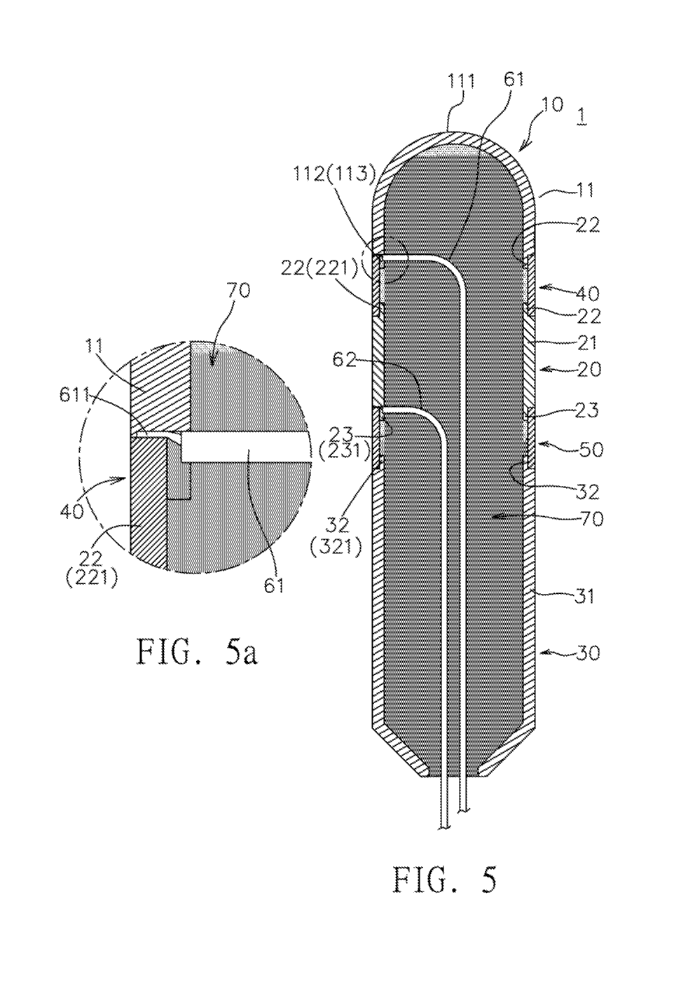

With reference to FIGS. 5 and 5a for the electrical treatment probe for caval sphincter in accordance with this disclosure, an electrical treatment probe casing (not labeled) is formed, and the electrical treatment probe casing is placed in a mold (not shown in the figure), and then an injection molding of a filler 70 is performed in the mold to fill the filler 70 into the electrical treatment probe casing to form the electrical treatment probe 1. Now, the electrically conductive portion 611 and the electrically conductive portion 621 are pressed by the conductive rings 40, 50 respectively, and the melted filler 70 has a higher temperature, so that the electrically conductive portions 611, 621 are embedded into the probe-head internal connecting tube 112 and the second internal connecting tube 23 respectively, while the electrically conductive portions 611, 621 and the conductive rings 40, 50 produce an excellent contact and positioning effect. In the aforementioned injection molding process, an appropriate pushing force (pressure) is preferred, so that the conductive ring 40 can be closely attached and combined between the head covering member 11 and the middle-section tube 21. As a result, the conductive ring 50 is closely attached and combined between the middle-section tube 21 and the probe-tail tube 31.

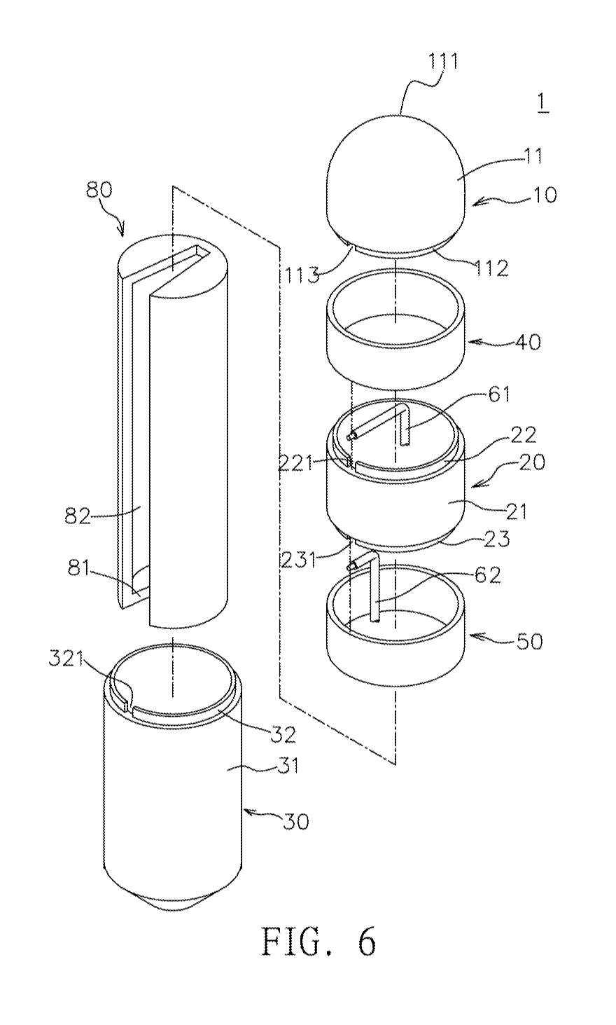

With reference to FIG. 6 for an electrical treatment probe structure for caval sphincter in accordance with the second preferred embodiment of this disclosure, this embodiment is based on the structure of the first preferred embodiment to make minor changes, and their difference resides on that this preferred embodiment further comprises a reinforcing member 80, and the reinforcing member 80 is put into the electrical treatment probe 1 in advance during the injection molding process, and then the injection molding process is performed to fill the reinforcing member 80 and the filler 70 into the electrical treatment probe 1. The reinforcing member 80 is substantially a cylindrical member having an opening 81, a cavity 82 communicated with the interior of the opening 81, so that when the filler 70 is filled into the cavity 82 during the injection molding process, the reinforcing member 80 provides a better support to the electrical treatment probe 1 to improve the overall strength of the electrical treatment probe 1.

The electrical treatment probe of this disclosure comes with a good design of the manufacturing components and assemblies to improve the convenience and cost-effectiveness of the manufacture of the electrical treatment probe and also overcomes the issue of permeating pigments of the welding material to the surface electrical treatment probe of the during the injection molding process to enhance the quality of the electrical treatment probe so formed.

What is claimed is:

1. An electrical treatment probe for caval sphincter, comprising:
a head portion, including a head covering member formed by an internal hollow tube, an arc end portion disposed at a front of the head covering member, and a probe-head internal connecting tube with a smaller diameter and coupled to a rear of the head covering member;
a middle portion, including a hollow middle-section tube, and a first internal connecting tube and a second internal connecting tube with a smaller diameter and coupled to the front and rears of the middle-section respectively;
a tail portion, including a hollow probe-tail tube, and a probe-tail internal connecting tube with a smaller diameter and coupled to the front of the probe-tail tube;
two conductive rings, one of the conductive rings being sheathed on the probe-head internal connecting tube and the first internal connecting tube of the head covering member, and the other conductive ring being sheathed on of the second internal connecting tube and the probe-tail internal connecting tube of the tail portion;
a conductive wire module, including a first conductive wire and second conductive wire, and the first conductive wire being pressed by one of the conductive rings to produce a contacting and positioning effect, and the second conductive wire being pressed by the other conductive ring to produce a contacting and positioning effect;
such that an electrical treatment probe casing is formed by the aforementioned elements, and the electrical treatment probe casing includes an injection molding installed therein and filled with a filler, and after the conductive ring is installed, the electric treatment probe casing, the head covering member, the middle-section tube and the probe-tail tube constitute a smooth surface of the electrical treatment probe.

2. The electrical treatment probe for caval sphincter according to claim 1, wherein the probe-head internal connecting tube of the head portion has a notch.

3. The electrical treatment probe for caval sphincter according to claim 1, wherein both of the first internal connecting tube and the second internal connecting tube have a notch separately.

4. The electrical treatment probe for caval sphincter according to claim 1, wherein the probe-tail internal connecting tube of the tail portion has a notch.

5. The electrical treatment probe for caval sphincter according to claims 2, wherein the first conductive wire is passed into the notch of the probe-head internal connecting tube or the notch of the first internal connecting tube.

6. The electrical treatment probe for caval sphincter according to claim 3, wherein the first conductive wire is passed into the notch of the probe-head internal connecting tube or the notch of the first internal connecting tube.

7. The electrical treatment probe for caval sphincter according to claims 3, wherein the second conductive wire is passed into the notch of the second internal connecting tube or the notch of the probe-tail tube.

8. The electrical treatment probe for caval sphincter according to claim 4, wherein the second conductive wire is passed into the notch of the second internal connecting tube or the notch of the probe-tail tube.

9. The electrical treatment probe for caval sphincter according to claim 1, wherein the first conductive wire has an electrically conductive portion embedded into the probe-head internal connecting tube of the head portion or the first internal connecting tube of the middle-section tube.

10. The electrical treatment probe for caval sphincter according to claim 1, wherein the second conductive wire has an electrically conductive portion embedded into the second internal connecting tube of the middle-section tube or the probe-tail internal connecting tube of the probe-tail tube.

11. The electrical treatment probe for caval sphincter according to claim 1, wherein the electrical treatment probe casing includes a reinforcing member installed therein.

12. The electrical treatment probe for caval sphincter according to claim 11, wherein the reinforcing member has an opening communicated with a cavity in the reinforcing member, and the cavity is filled with the filler.
